(19) United States
(12) Patent Application Publication
(10) Pub. No.: US 2007/0239119 A1
(43) Pub. Date: Oct. 11, 2007

(54) Adjustable curved needle

(76) Inventor: Eugene G. Lipov, Chicago, IL (US)

Correspondence Address: BRIAN J. LUM;ICE MILLER LLP, SUITE 3500, 200 WEST MADISON STREET, CHICAGO, IL, 60606, US

(21) Appl. No.: 11/374,491

(22) Filed: Mar. 13, 2006

(51) Int. Cl. A61M 5/32; A61M 25/00

(52) U.S. Cl. 604/272; 604/528; 604/530

(57) ABSTRACT

An adjustable curved needle having a flexible cannula with a linear configuration and a relatively inflexible stylet with a curved configuration. Insertion of the stylet into the cannula, causes the configuration of the cannula to conform to the configuration of the stylet, such that the configuration of the needle may be adjusted by insertion and withdrawal of the stylet from the cannula.

### BACKGROUND OF THE INVENTION

The present invention relates to medical instruments and especially to an adjustable curved needle for use in nucleoplasty and other medical procedures.

Herniated or slipped discs are a common cause of back pain. The spine is made of a series of stacked vertebrae that form a tube which holds and protects the spinal cord. Adjacent vertebrae are separated by discs that serve to cushion and absorb pressure between the vertebrae. The discs comprise an annulus or outer wall made of tough, fibrous tissue that surrounds an inner nucleus made of gelatinous material. Nerve roots branch off on either side of the spinal cord near the discs and exit the spine through the spaces between adjacent vertebra.

When excessive pressure is placed on the disc, the annulus may bulge abnormally or rupture and allow the nucleus to be forced outward, producing a herniated disc. If the annulus or nuclear material presses on the nerve root or spinal cord, it can cause pain, weakness or numbness. This condition can be corrected by nucleoplasty, in which sufficient material is removed from the nucleus to relieve the pressure and allow the disc to withdraw into its normal position. Percutaneous disc nucleoplasty is a minimally invasive procedure, in which a needle is inserted into the herniated disc from the patient's back for removal of nuclear material. However, because of the architecture of the spine and vertebrae, it can be difficult to access the disc without risk of damage to the spinal cord or nerve root. Particular skill is required where the herniated disc is located near the base of the spine, at the L5/S1 vertebrae.

Accordingly, there is a need for an instrument that permits the user to more easily access the disc and avoid the risk of damage to the spinal cord and nerve root.

### BRIEF SUMMARY OF THE INVENTION

These needs and other needs are satisfied by the present invention, which comprises an adjustable needle, having a flexible cannula with a bore and a stylet sized to be inserted into the bore. The cannula has a first configuration and the stylet has a second configuration different from the first. Inserting the stylet into the cannula deflects the cannula from the first configuration and causes the cannula to conform to the second configuration.

### BRIEF DESCRIPTION OF THE DRAWINGS

FIG. 1a is side elevation view of a needle comprising a cannula and stylet.

FIG. 2 is a detail perspective view of the tips of the cannula and stylet of FIG. 1.

FIG. 3 is a side elevation view of the stylet and cannula of FIG. 1, the stylet partially inserted into the cannula.

FIG. 4 is a side elevation view of the stylet and cannula of FIG. 1, the stylet fully inserted into the cannula.

FIG. 5 is a detail perspective view of the tip of the stylet and cannula of FIG. 4.

### DETAILED DESCRIPTION OF THE INVENTION

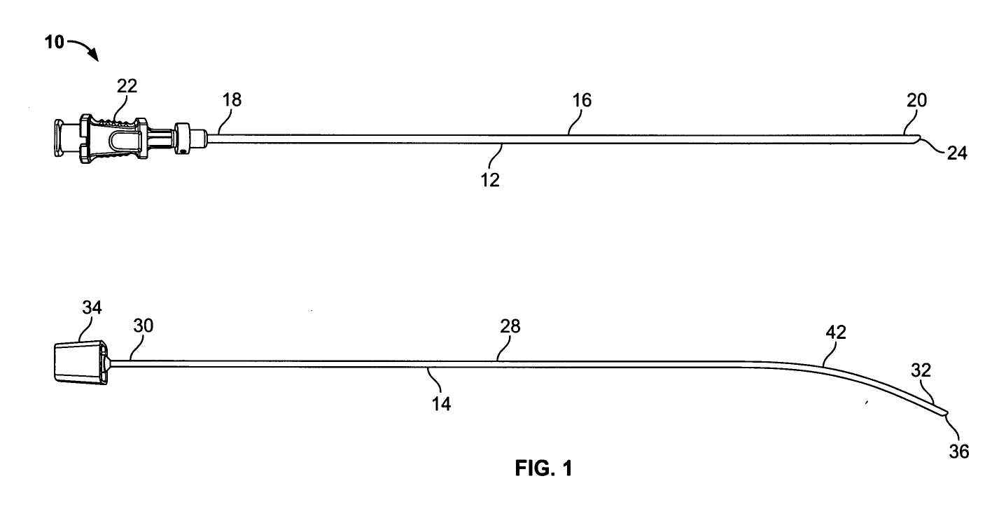

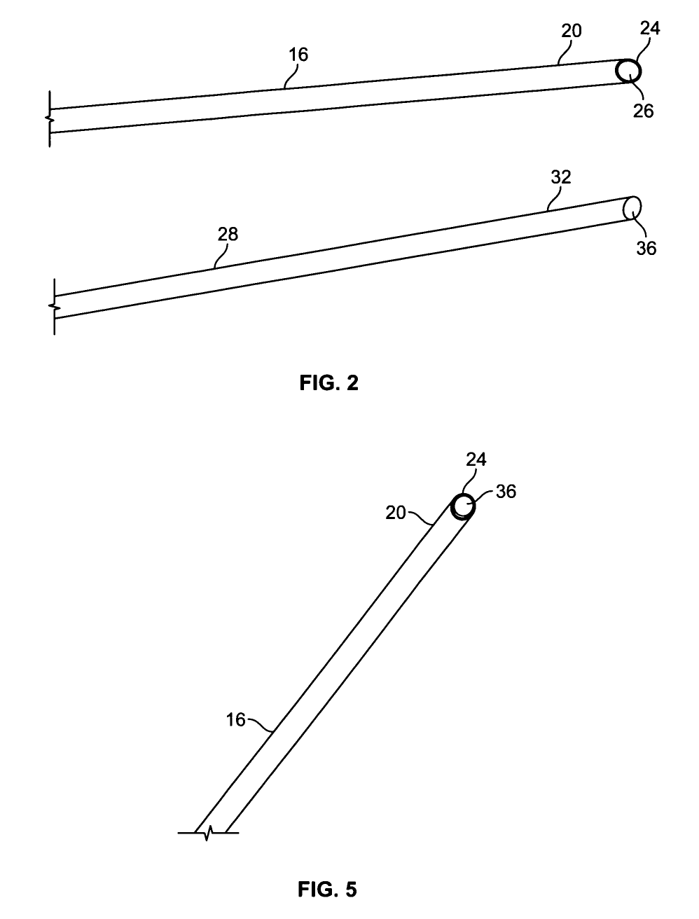

Referring to FIGS. 1-5, an adjustable curved needle 10 is described, comprising a flexible cannula 12 and stiff, curved stylet 14. As shown in FIG. 1, cannula 12 has a conventional configuration with a linear body 16 having ends 18, 20. End 18 is provided with a connector for attaching cannula 12 to other devices, such as a conventional female luer connector 22. End 20 has a tip 24. As best shown in FIG. 2, a bore 26 travels through the length of cannula 12. In addition, the outer surface of cannula 12 may be printed with indicia, such as bands or other markings (not shown) that indicate the length of the needle.

When used for spinal disc nucleoplasty, cannula 12 preferably has a length of about 6 inches and an outer diameter equivalent to a 17 gauge needle. However, those of skill in the art will appreciate that the length and outer diameter of cannula 12 may vary according to the specific application. Tip 24 is preferably beveled to facilitate penetration of tough tissue, but may be blunt or have other configurations known in the art, as may be required by the specific application.

Body 16 of cannula 12 is made of a relatively flexible material that can readily be deflected from its linear configuration. In a preferred embodiment, body 16 is made of a material that has a memory, such that body 16 tends to substantially return to its linear configuration after it has been deflected. Exemplary materials include thin-walled stainless steel.

As shown in FIG. 1, stylet 14 has a shaft 28 with a curved configuration having ends 30, 32. Shaft 28 is sized to be inserted through connector 22 and into the bore 24 of cannula 12. End 30 of stylet 12 is provided with a cap 34 that is sized and shaped to receive connector 22. End 32 has a tip 36. Cap 34 serves as a handle for insertion and withdrawal of stylet 14 from cannula 12, and as stop to prevent further insertion of the stylet into the cannula.

In a preferred embodiment, shaft 28 is sufficiently long, such that when stylet 14 is fully inserted into cannula 12, cap 34 encloses connector 22 and shaft 28 extends completely through the length of cannula 12. In a further preferred embodiment, tip 36 of stylet 14 is shaped to conform to the configuration of tip 24 of cannula 12, such that tips 24 and 36 form a continuous surface when stylet 14 is fully inserted into cannula 12. For example, FIG. 5 shows a continuous beveled surface formed by the combination of a beveled tip 24 of cannula 12 and a beveled tip 36 of stylet 14.

When used for nucleoplasty, it is preferred that the curved configuration of shaft 28 comprises a single curve having an interior angle of between 90° to 180°, and most preferably about 160°. The apex of the curve is positioned at about 0.5-2 inches from tip 36 of shaft 28 and most preferably about 1 inch from tip 36. However, different users may have a preference for different curved configurations.

Shaft 28 of stylet 14 is sufficiently inflexible such that, when stylet 14 is inserted into cannula 12, body 16 of the cannula is deflected from its original linear configuration and conforms to the curved configuration of shaft 28. In a preferred embodiment, shaft 28 is made of a relatively stiff material, such as solid stainless steel. Those of skill in the art will appreciate that the stiffness of shaft 28 depends in part on its length and thickness, and that shaft 28 may be hollow and/or may be made of a variety of other materials depending upon its configuration. It is further preferred that cannula 12 and stylet 14, including cap 34 and connector 22, be made of materials that can withstand sterilization by various means known in the art, such as stainless steel and autoclavable plastics.

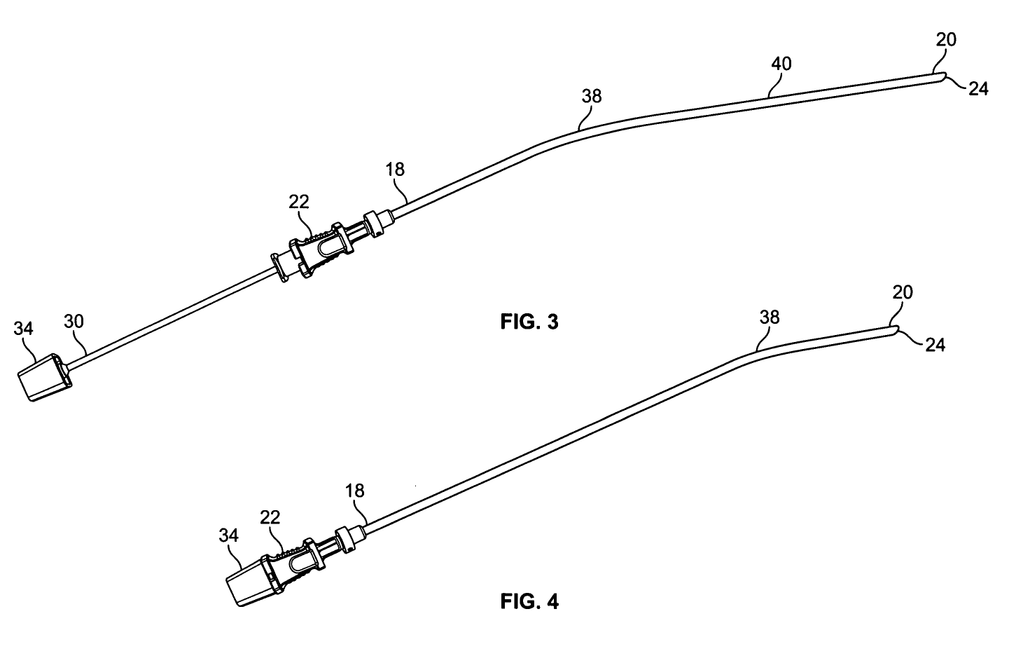

As shown in FIGS. 3 and 4, the configuration of needle 10 is adjusted by inserting or withdrawing shaft 28 of stylet 14 from bore 24 of cannula 12. FIG. 3 shows stylet 14 partially inserted into bore 24, which deflects the linear configuration of cannula 12 and causes it to conform to the curved configuration of the stylet. As shaft 28 is inserted into cannula 12, the curved configuration 38 travels along the length of cannula 12—i.e. as shown by comparison of FIGS. 3 and 4. As shaft 28 is withdrawn from cannula 12, the memory characteristics of the cannula cause the portion of body 16 from which shaft 28 is withdrawn to substantially return to its original linear configuration 40.

In a preferred embodiment, the outside surface of the elbow or apex 42 of the curved configuration of shaft 28 may be eased by forming a flattened surface (not shown) to facilitiate the insertion and withdrawal of stylet 14 from cannula 12. Other methods of facilitating the insertion and withdrawal stylet 14 from cannula 12 may also be used, such as coating the stylet with polytetrafluoroethylene, silicone or other lubricant.

Adjustable needle 10 may be used to address regions within the spine that are not directly accessible using a conventional straight needle because of the intervening architecture of the spine or the risk of damage to adjacent features, such as the spinal cord or nerve root. For example, when used for nucleoplasty, needle 10 is typically inserted into the patient at a location and/or in an orientation that avoids any intervening architecture or features. Once inserted safely past such architecture or features, the configuration of needle 10 is then adjusted to redirect tip 24 of the cannula toward the target disc by inserting and/or withdrawing stylet 14 from cannula 12. After needle 10 engages the target disc, stylet 14 is then completely withdrawn from cannula 12 to permit the removal or nuclear material. Those of skill in the art will appreciate that adjustable needle 10 may be applied to other medical procedures where the target cannot be directly addressed. Furthermore, although stylet 14 is described and illustrated herein as having a simple curved configuration, other more complex configurations are possible—e.g., an S-curve configuration—as may be required by the specific application.

It will be apparent to those of skill in the art that changes and modifications may be made in the embodiments illustrated herein, without departing from the spirit and scope of the invention.

What is claimed is:

1. An adjustable needle, comprising:
a flexible cannula with a bore, said cannula having a first configuration;
a stylet sized to be inserted into said bore, said stylet having a second configuration that is different from said first configuration;
wherein said cannula is deflected from said first configuration and conforms to said second configuration when said stylet is inserted into said bore.

2. The adjustable needle of claim 1, wherein said cannula tends to return to said first configuration when said stylet is withdrawn from said bore.

3. The adjustable needle of claim 1, wherein said first configuration is linear and said second configuration includes at least one curve, said cannula conforming to said curve when said stylet is inserted into said bore.

4. The adjustable needle of claim 3, wherein said curve forms an interior angle of between about 90° to about 180°.

5. The adjustable needle of claim 4, wherein said interior angle is about 160.

6. The adjustable needle of claim 3, wherein said stylet has a flattened surface at said curve.

7. The adjustable needle of claim 3, wherein said stylet has a tip, said curve positioned proximal to said tip.

8. The adjustable needle of claim 7, wherein said curve is positioned between about 0.5 to about 2 inches from said tip.

9. The adjustable needle of claim 8, wherein said curve is positioned about 1 inch from said tip.

10. The adjustable needle of claim 1, wherein said cannula has a first tip and said stylet has a second tip, said first and second tips forming a continuous surface when said stylet is inserted onto said bore.

11. The adjustable needle of claim 10, wherein said continuous surface is a bevel.

12. The adjustable needle of claim 1, wherein said stylet is coated with a lubricant.

13. The adjustable needle of claim 1, wherein said cannula is made of thin-walled steel.

14. An adjustable needle, comprising:
a flexible cannula with a bore, said cannula having a linear first configuration;
a stylet sized to be inserted into said bore, said stylet having a second configuration that is different from said first configuration and that includes at least one curve;
wherein said cannula is deflected from said first configuration and conforms to said curve when said stylet is inserted into said cannula, and wherein said cannula tends to return to said first configuration when said stylet is withdrawn from said cannula.

15. The adjustable needle of claim 14, wherein said curve forms an interior angle of between about 90° to about 180°.

16. The adjustable needle of claim 15, wherein said interior angle is about 160°.

17. The adjustable needle of claim 14, wherein said stylet has a flattened surface at said curve.

18. The adjustable needle of claim 14, wherein said stylet has a tip, said curve positioned between about 0.5 to about 2 inches from said tip.

19. The adjustable needle of claim 18, wherein said curve is positioned about 1 inch from said tip.

20. An adjustable needle, comprising:
a flexible cannula with a bore, said cannula having a linear first configuration;
a stylet sized to be inserted into said bore, said stylet having a second configuration that is different from said first configuration and that includes at least one curve; and
a flattened surface on said stylet at said curve;
wherein said cannula is deflected from said first configuration and conforms to said curve when said stylet is inserted into said cannula, and wherein said cannula tends to return to said first configuration when said stylet is withdrawn from said cannula.
